(19) United States
(12) Patent Application Publication
(10) Pub. No.: US 2006/0111284 A1
(43) Pub. Date: May 25, 2006

(54) Method for treating cancer

(76) Inventors: Edward J. Yurkow, Hillsborough, NJ (US); Fred H. Mermelstein, Newton, MA (US)

Correspondence Address: LICATLA & TYRRELL P.C., 66 E. MAIN STREET, MARLTON, NJ, 08053, US

(21) Appl. No.: 10/522,092

(22) PCT Filed: Jul. 30, 2003
(86) PCT No.: PCT/US03/23867

§ 371 Date: Oct. 19, 2005

(30) Foreign Application Priority Data
Aug. 26, 2002 (US) 10/228,644

(51) Int. Cl. A61K 38/17

(52) U.S. Cl. 514/12

(57) ABSTRACT

A method of treating lymphoma, ovarian cancer, colorectal cancer, or gastric cancer by administering an effective amount of Mesna to a patient is provided. A method for treating and reducing the effective dose of an anti-cancer agent by administering Mesna in conjunction with an anti-cancer agent is also provided.

### BACKGROUND OF THE INVENTION

Sodium-2-mercaptoethane sulphonate, known as Mesna, was approved for the prevention of the urotoxic effects of oxazaphosphorine cytotoxic agents, cyclosphosphamide and ifosfamide. Mesna is traditionally given to patients undergoing chemotherapy, to offset the toxic effects of chemotherapy on the bladder. Mesna is pharmacologically and toxicologically a relatively inert substance, even in very high dose, i.e. 1000 mg/kg. It is particularly striking that even high Mesna doses do not interfere with the curative potency of the oxazaphosphorines or cytostatics.

In the body Mesna is rapidly inactivated to form inert Mesna disulfide (diMesna). Within a few minutes after administration, more than 90% of the administered dose has been transformed into Mesna disulphide which remains in the vascular system and is rapidly eliminated via the kidneys. After glomerular filtration, a considerable part of Mesna disulphide is reduced to a third compound which reacts and detoxifies the urotoxic oxazaphosphorine metabolites in the urine. The reaction with glutathione plays a particular role in the reduction of Mesna disulphide to Mesna in the renal epithelia, suggesting a three stage reaction. The first reactions are catalyzed by thiol transferase, the last by glutathione reductase.

Recent studies have focused on the prevention of the urotoxic effects of chemotherapeutic drugs in patients when Mesna is administered in conjunction with conventional chemotherapeutic drugs, for example, Russo, 2000 P. Seminars in Oncology 27(3):284-98. Further, Blomgren et al. Meth Find Exp Clin Pharmacol 1990 12(10) 691-697 disclose that certain cell lines, such as T24 and HU549, in culture seem to support the observation that patients with superficial bladder cancer benefit from Mesna treatment. Blomgren et al. further disclose however, that patients with tumor locations other than the bladder are unlikely to benefit from Mesna treatment because of the rapid disulfide formation of Mesna in the blood.

U.S. Pat. Nos. 6,025,488 and 6,066,645 disclose DiMesna (Disodium-2,2′-dithiobisethane sulfonate) derivatives thereof that have been found to selectively reduce the toxicity of certain antineoplastic agents, namely certain platinum complex drugs in vivo. DiMesna and its disulfide analogues and derivatives have been shown to enhance the antineoplastic activity of some platinum complexes. U.S. Pat. No. 5,019,596 discloses that administration of sodium mercaptoethansulphonate causes the normalization of the urinary levels of tryptophan and its urinary metabolites in patients suffering from bladder carcinoma. Derivatives of mercapthansulphonic acid are active in the therapy of the bladder carcinoma and in the therapy and prevention of the cistinic kidney calculi.

The present invention provides a method of treating lymphoma, ovarian cancer, colorectal cancer, or gastric cancer comprising administering an effective amount of Sodium-2-mercaptoethane sulphonate to a patient with lymphoma, ovarian cancer, colorectal cancer or gastric cancer.

### SUMMARY OF THE INVENTION

The present invention provides a method of treating lymphoma, ovarian cancer, colorectal cancer, or gastric cancer comprising administering an effective amount of Sodium-2-mercaptoethane sulphonate (Mesna) to a patient with lymphoma, ovarian cancer, colorectal cancer or gastric cancer.

The present invention further provides a method of reducing the effective dose of an anti-cancer agent comprising administering Mesna in conjunction with an anti-cancer agent.

The present invention further provides a method of treating cancerous tumors comprising administering an effective dose of an anti-cancer agent in conjunction with a redox clamping agent.

### BRIEF DESCRIPTION OF THE DRAWINGS

FIG. 1 shows the redox clamping function of Mesna in human LNCaP cells.

FIG. 2 shows the chemosensitizing properties of a redox clamping agent co-administered with an anti-cancer agent.

### DETAILED DESCRIPTION OF THE INVENTION

DiMesna is absorbed from the gastrointestinal tract and undergoes reduction to Mesna during or after absorption. It has been found that Mesna actually kills cancerous cells in patients with extensive metastatic disease. Surprisingly, Mesna has been found to exhibit chemotherapeutic anti-tumoral activity in cancer patients suffering from certain types of cancers. Mesna has also been found to be effective in patients who are resistant to chemotherapy and who have had full dose radiation delivered with poor prognosis based upon results of traditional therapeutic agents for cancer.

The present invention provides a method of treating lymphoma, ovarian cancer, colorectal cancer, or gastric cancer comprising administering an effective amount of Mesna to a patient with lymphoma, ovarian cancer, colorectal cancer or gastric cancer. Mesna inhibits the growth of several human tumor cell lines in culture, and there are no strict relationships between the histopathological origin of the cell lines and the sensitivity of the cell lines to Mesna. Mesna inhibited incorporation of 3H-thymidine, proline and uridine to achieve effective treatment concentration in humans, or redox clamping, it is preferred that Mesna be administered to a patient in a dose of 600 mg/m² of Mesna four times per day, or up to any dose equal to the dose limiting toxicity. It is further preferred that the patient be administered Mesna for a seven to ten day period. The doses may be cycled. The actual dose will be dependent on the size, type and location of the tumor as well as the overall status of the patient. It is preferred that high concentrations of Mesna be administered intravenously to achieve clamping initially, with further administration of oral doses to maintain the clamped state. Tumor cells have been found to be reduced by approximately 15% TO 30% after 1 treatment of Mesna. However, Dimesna did not interfere with the growth inhibitionary activity of Mesna.

Cancer cell lines were growth inhibited when Mesna was administered on several consecutive days. Mesna has shown good activity in preventing the urotoxicity of oxazaphosphorine compound. Surprisingly, it has now been found that Mesna inhibits growth of several human tumor cell lines, namely lymphoma, ovarian cancer, colorectal cancer, and gastric cancer, in culture when the drug is administered on several consecutive days.

In one aspect, the present invention further provides a method of using a redox clamping agent as a chemosensitizer comprising contacting tumor cells with a specific class of cytotoxic agent for a period of time, preferably between 2 and 4 hours, and then washing the agent from the cells and administering the redox clamping agent. It is preferred that the redox clamping agent is Mesna. The cytotoxic or anti-cancer agents used in this design would be those such as arsenic, hydrogen peroxide and other anti-cancer agents that are known to induce a cytotoxic effect by interacting with, depleting or sequestering cellular thiols. During the two to four hour treatment these anti-cancer agents deplete cellular thiols and induce apoptotic mechanisms in the cells. However, the stress-response that is induced in the cells by these anti-cancer agents also up-regulates the production of antioxidants such as glutathione and metallothionein. These antioxidants function as potent inhibitors of apoptosis. If redox clamping agents are added after the apoptotic mechanisms are initiated, but prior to the antioxidant rebound, the redox clamping agents will function in two ways to enhance the cytotoxic effects of the anti-cancer agents. It is preferred that the redox clamping agent be Mesna, which is a thiol with low reducing potential. First, the redox clamping agents will permit the progression of apoptotic signals and the mechanisms required to mediate cell death. Secondly, the redox clamping agents will promote the apoptotic response by dramatically attenuating the antioxidant “rebound” that is associated in the inhibition of apoptosis. Redox clamping agents chemically interact with the anti-cancer agent or oppose aspects of the early stress-response that is induced by pure oxidants. Therefore, a two-step treatment protocol is required.

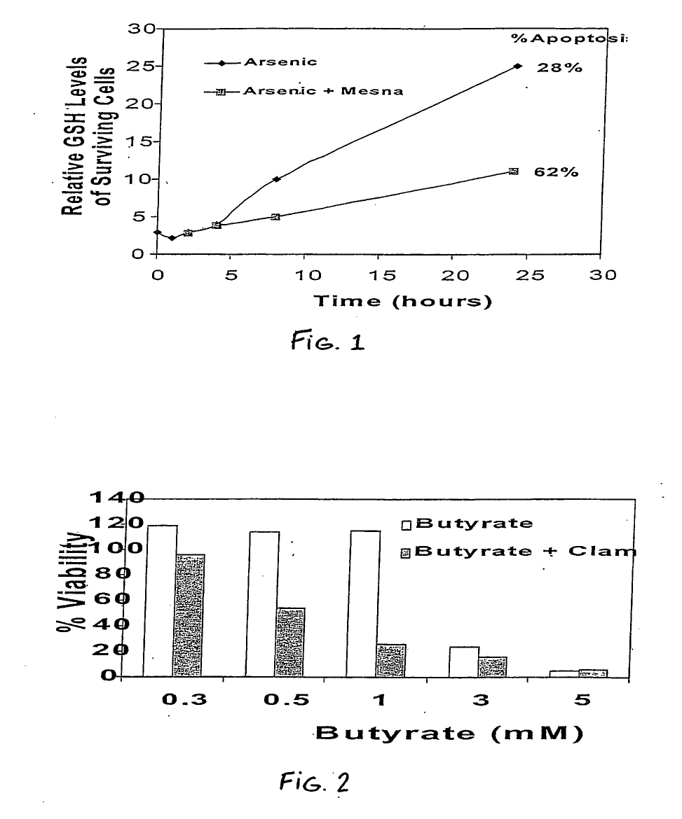

Human LNCaP cells, treated with 100 uM arsenic for 2 hours then washed and re-fed with normal medium, develop an elevation in cellular glutathione levels over a 24-hour period. Since exogenous antioxidants are known to be potent inhibitors of apoptotic mechanism, it is believed that internally-produced antioxidants such as glutathione function in a similar manner. As shown in FIG. 1, treatment of the human LNCaP cells with arsenic resulted in the apoptosis of 28% of the cells. However, when the arsenic treated cells were washed and incubated in medium containing 100 uM Mesna, the elevation in glutathione levels was dramatically diminished and the fraction of apoptotic cells increased more than two-fold compared to arsenic alone. Although Mesna increases apoptosis alone, the low concentrations of Mesna utilized in this two-step treatment protocol increased apoptosis minimally i.e., to 11 percent from a background of 3 to 5 percent in untreated cells.

To further exemplify the redox clamping properties of Mesna, stress was induced in H4 Rat Hepatoma cells by treatment with 100 uM hydrogen peroxide for two hours and then the medium was removed and replaced with an anti-cancer agent. The cellular viability, as well as glutathione and metallothionein levels were determined after 24 hours in culture. Mesna as well as DMSA were found to oppose stress-induced upswings in antioxidant levels. The inhibition of stress induced upswing in antioxidant levels is characteristic of redox clamping agents.

The present invention further provides a method of reducing the effective dose of an anti-cancer agent necessary to be effective against cancer comprising administering a redox clamping agent in conjunction with an anti-cancer agent. It is preferred that the redox clamping agent is Mesna or DMSA. Redox clamping agents have the ability to maintain cells in a selected redox state. Redox clamping agents do not permit the cell to successfully compensate for treatment-induced alterations in cellular redox status. Redox clamping agents are useful in enhancing the therapeutic activity of chemotherapeutic agents such as butyrate that are dependent upon the redox state of the cell. Redox clamping agents are further useful in controlling hyperproliferation of cells and conditions associated with abnormal fluctuations in the redox state of cells. In one aspect, this invention provides a method of treating cancerous tumors comprising administering an effective dose of an anti-cancer agent in conjunction with a redox clamping agent, wherein the redox clamping agent acts as a chemoenhancer or a chemosensitizer. When used in conjunction or combination with an anti-cancer agent, it has been found that Mesna acts as a chemoenhancer. The administration of Mesna in conjunction with an anti-cancer agent may be as separate compounds or in a composition formulated and titrated to a dose and in a concentration that would achieve the optimal therapeutic dose of the anti-cancer agent providing a higher tumor cell kill with a lower dose of the anti-cancer agent than the typical therapeutic dose administered to a patient. Administration of Mesna in conjunction with an anti-cancer agent demonstrates the redox clamping and chemosensitizing properties of Mesna. The cytotoxic or anti-cancer agents utilized induce an apoptotic mechanism that does not involve a pure oxidative stress. Therefore, inhibition of the initial apoptotic signals by the presence of the redox clamping agent is not a factor. Typical examples of anti-cancer agents include: apoptosis inducing agents, differentiating agents, DNA intercalating agents, and alkylating agents. For the anti-cancer agents such as butyrate, redox clamping agents stabilize the redox state keeping cellular levels of reduced glutathione and possibly other antioxidant levels low. As shown in FIG. 2, Butyrate induces apoptosis in cells at high concentrations, namely those concentrations greater than 3 mM. However, in the presence of a redox clamping agent, an effective “kill” can be demonstrated with concentrations of butyrate as low as 0.5 mM. Mesna and DMSA are preferred redox clamping agents. Similar chemosensitizing effects of redox clamping agents on chemotherapeutic agents such as adriamycin have also been observed. A therapeutic human dose may be extrapolated from effective concentrations derived from in vitro data, including animal and cell culture experiments, to achieve redox clamped state in humans. It is believed that an effective human dose range of oral administration of Mesna as a redox clamping agent or chemosensitizer is between 1 gram/m²/day to 24 grams/m²/day or up to any dose equal to the dose limiting toxicity. It is preferred that the Mesna be administered in 2 to 3 fractionated doses each day, for a period of seven to ten days. The actual dose will be dependent on the size, type and location of the tumor, as well as the overall status of the patient as indicated by results of clinical chemistry. A clamped state would then be maintained by continued dosing. It may be advisable to achieve clamping initially with high concentrations of Mesna administered intravenously and then maintain the clamped state with oral dosing of Mesna. This combination dosing of Mesna and an anti-cancer agent is important for anti-cancer agents which have dose limiting adverse effects and toxicity profiles, as both the adverse effects and toxicity of the agent are reduced due to the combination dosing of Mesna and an anti-cancer agent. Thus, the combination dosing of Mesna in conjunction with an anti-cancer agent permits a lower dose of the anti-cancer agent to be used.

The following nonlimiting examples are provided to further illustrate the present invention.

### EXAMPLE 1

From December 1996 to March 1998 a study was performed on 14 ambulatory patients with metastatic and/or relapsed tumors refractory to conventional therapy. Eligibility criteria included histologically-confirmed advanced cancer, patients age was greater than 18 years, with a life expectancy no less than two months. The patients had not undergone major surgery within 2 weeks, or radiotherapy and/or chemotherapy within one month of the study. All patients had adequate hematopoietic (absolute neutrophil count above 1500/ml and platelet count above 100,000/ml) hepatic (total bilirubin level below 1.5 mg./dl) and renal (creatine concentration below 1.5 mg/dl) creatine clearance above 60 ml/min) functions. Exclusion criteria included active uncontrolled infection, pregnancy, lactation and any other co-existing medical problems severe enough to prevent full compliance with the study. Objectively measurable disease in the patients was required. All patients gave informed written consent before treatment. 600 mg/m² of Mesna was given by oral route four times a day, masked in orange juice for 10 consecutive days. Cycles were repeated every 15 days. No simultaneous chemotherapy and or radiotherapy and no other supportive care was given. The main objective of the study was to determine the anti-tumoral activity and toxicity of Mesna for heavily and or refractory pretreated cancer patients. The study group included one Non-Hodgkin lymphoma patient, one Hodgkin lymphoma patient, one colorectal cancer patient, one small cell lung cancer patient two non-small cell lung cancer patients, one patient exhibiting cancer of the cervix, one melanoma patient, two ovarian cancer patients, one bladder cancer patient, one soft tissue sarcoma patient, one gastric cancer patient and one patient with skin metastases of unknown origin. The patients were all previously treated with between two and four different chemotherapy regimens. Mesna was administered as a single agent to all of the patients in the study. The toxic effects of Mesna and number of nodes were assessed during the first cycle of Mesna administration. Two patients with ovarian cancer reduced ascites and pain on occasion of receiving second and third course of treatment respectively. For colorectal cancer a greater than 50% reduction of nodular lesions metastasized to liver was registered, during the second schedule of therapy. For gastric cancer a greater than 50% reduction of a subcutaneous nodule was measured during the second course of Mesna treatment. No objective responses were achieved for patients with lung cancer, soft tissue sarcoma, skin metastases of unknown origin, bladder cancer, melanoma and cancer of the cervix.

### EXAMPLE 2

In all of the experiments associated with the investigation of Mesna as a redox clamping agent, as a stand-alone anticancer agent as well as a chemosensitizer, the cell culture medium used was Swim's S-77 medium containing 12 uM cystine, instead of conventional levels of this amino acid (i.e., 50 uM). All other components of the medium were maintained at conventional concentrations. The reason for the decreased cystine of the medium is based on the observation that cultured cell lines, maintained in normal culture medium (containing approximately 50 uM cystine), exhibit supra-physiological levels of reduced glutathione compared to primary cell lines as well as to the already-elevated levels of glutathione in cells freshly isolated from tumors. The ability of cystine to supply cells with cysteine, which then function as a precursor amino acid for glutathione synthesis, is thought to be responsible for the abnormally high basal levels of glutathione in cultured tumor cell lines. In the present invention it was found that by adjusting the cystine concentration to 12 uM, the glutathione levels of tumor cell lines were maintained at levels similar to those in cells isolated from actual tumors.

What is claimed:

1. A method of treating lymphoma, ovarian cancer, colorectal cancer, or gastric cancer comprising administering an effective amount of Mesna to a patient with lymphoma, ovarian cancer, colorectal cancer or gastric cancer.

2. A method of reducing the effective dose of an anti-cancer agent comprising administering a redox clamping agent in conjunction with an anti-cancer agent.

3. The method of claim 2 wherein the anticancer agent is Mesna.

4. A method of treating cancerous tumors comprising administering an effective dose of an anti-cancer agent in conjunction with a redox clamping agent, wherein the redox clamping agent acts as a chemoenhancer or a chemosensitizer.
